(19) United States
(12) Patent Application Publication
(10) Pub. No.: US 2019/0139395 A1
(43) Pub. Date: May 9, 2019

(54) HYGIENE MONITORING SYSTEM

(71) Applicant: Nesa Solutions, Inc., Miami, FL (US)

(72) Inventors: Atanas Dimitrov Rogachev, Plovdiv (BG); Neil Salem, Miami, FL (US); Helmer van der Veer, Miami, FL (US); Albert Nahmad, Aventura, FL (US)

(73) Assignee: Nesa Solutions, Inc., Miami, FL (US)

(21) Appl. No.: 16/156,855

(22) Filed: Oct. 10, 2018

Related U.S. Application Data
(60) Provisional application No. 62/570,157, filed on Oct. 10, 2017.

(51) Int. Cl. G08B 21/24; A47K 5/12

(52) U.S. Cl. CPC G08B 21/245; G16H 40/20; A47K 5/1217

(57) ABSTRACT

A hygiene monitoring system is provided, where the system includes a location sensor that determines the location of a user within a healthcare area, hygiene zone and/or patient zone; customizable, predefined rules that determine when a hand washing event is required based upon to the location of that user or relative location of multiple users; and a hand washing register and a soap level meter in communication to determine whether a hand washing event has occurred. The hand wash register can determine if a user is within proximity of the soap dispenser, can determine whether the user's hands are underneath the soap dispenser, and can receive an indication from the soap level meter to determine whether the soap dispenser has been used and/or if the soap dispenser was used by the user while within proximity of the soap dispenser. The system can collect the corresponding data and provide real-time alerting and reporting.

### CROSS-REFERENCE TO RELATED APPLICATION

This application claims priority to U.S. Provisional Patent App. No. 62/570,157 titled Hygiene Monitoring System, the disclosure of which is hereby incorporated by reference.

### FIELD

The subject matter of the present disclosure relates to hygiene. And more particularly to a hygiene monitoring system aimed at reducing the risk of infections in businesses, hospitals, and other public facilities.

### BACKGROUND

Personal hygiene is a critical factor in preventing the spreading of germs. Businesses are aware that keeping their employees' hands clean through improved hygiene is an important step that can be taken to reduce the risk of their employees getting sick and spreading germs to others.

Clean hands are even more important in medical facilities, such as hospitals. The prevention and control of healthcare-associated infection (HAI) is critical for a well-functioning health ecosystem. HAIs are infections that are contracted by patients while receiving treatment for medical or surgical conditions in a hospital or medical facility. Thousands of people become sick and/or die every day around the world from infections acquired while receiving health care. Hands are the main pathways of germ transmission during health care. Therefore, hand hygiene is a critical factor that must be considered in order to avoid the transmission of harmful germs and prevent health care-associated infections.

One personal hand hygiene device common to all businesses and health care facilities is the soap or alcohol dispenser. There are certain instances in the healthcare setting when using a soap or alcohol dispenser is important. These instances include, for example, before touching a patient, before any cleaning procedure, after body fluid exposure risk, after touching a patient, and after touching patient surroundings. It is important that healthcare workers wash their hands after each one of these instances. Yet there is no procedure in place to ensure compliance with proper hand hygiene during these instances or at any time.

Most common soap dispensers are not smart devices and are designed to simply dispense soap, alcohol or hand sanitizer. Most soap dispensers on the market do not include or integrate a control or monitoring system that determines when employees are required to use the soap dispenser, whether employees actually use or don't use the soap dispenser when required to do so, the amount of soap that is dispensed, and/or the amount of soap remaining in the soap dispenser. Nor are they able to accurately track in real time how often the soap dispenser is used by each employee when required and provide corresponding real-time alerting (if required) and reporting.

### SUMMARY

The present disclosure solves the aforementioned problems by providing a hygiene monitoring system that monitors and helps to improve personal hygiene and that allows for the monitoring, collection, and analysis of data in order to determine and improve the effectiveness of the hygiene monitoring system.

The hygiene monitoring system of the present disclosure includes, as at least some of its objectives, the ability to comply with a given hand hygiene framework, alert a medical facility user prior to any breaching of the framework, report on hand hygiene practices, and provide traceability and history of staff movements.

The integrity of these objectives is achieved through a variable combination of several elements such as, but not limited to, tracking tags worn by staff members and patients (e.g. an RFID tag), a hand wash register, a soap level meter, various sensors, and a computing device.

The frontend component of the computing device (i.e., the user interface) allows a customer or facility to define the parameters of the three areas (healthcare area, hygiene zone and patient zone), to define when a hygiene event is required, to dictate what alerting is required, to dictate when the alert is to be activated and what type of alert will be implemented, etc. The backend component of the computing device (i.e., the business rules, logic, geo-fencing capabilities, etc.) allows the system to set the parameters of the three areas (healthcare area, hygiene zone and patient zone), compiles all the data points that are transmitted from tracking tags worn by staff members and patients to the location sensors and to the computing device and, based upon the processing of that data, be able to deliver the real-time alerting and reporting.

The system can determine the position of a person (as well as the relative positions between multiple people), and use the corresponding location information to identify their positioning within one of three areas relating to hand washing compliance regulations: (i) the healthcare area, which can be defined as any area determined by a virtual floor plan in which hygiene washing is to be monitored (e.g., a patient room), within which contains a soap dispenser, (ii) the hygiene zone, which can be defined as a virtual geo cone of a specified diameter around the soap dispenser, and (iii) the patient zone, which can be defined as a dynamic, virtual geo cone with a specified diameter around a patient.

The system of the present disclosure can determine the requirement of a washing event by an end user or facility setting customized rules for hygiene events or washing events on the computing device.

The system of the present disclosure can determine the occurrence of a washing event. The system determines if a washing event was complied with through three mechanisms. First, the system uses information about the location of a person in a hygiene zone (potentially for a predetermined period of time) to infer that hand washing occurred. Second, the system uses a sensor on the hand wash register or soap, alcohol, or sanitizer dispenser to detect the presence of hands underneath the soap dispenser to infer that a washing event occurred. Third, the system monitors changes or decreases in the level of soap, alcohol, or sanitizer in the dispenser to infer if the washing occurred.

The hygiene monitoring system of the present disclosure is further equipped with a backend computing device which contains business rules and logic that enables the system to store and analyze the data collected in order to improve performance of the system, support inventory maintenance, as well as proactively influence human behavior by real time notification of any action supportive of any hand hygiene policy. Additionally, the computing device may also generate reports based on the tracked and gathered information. Data collected by the system includes location of persons and patients, the identity of the person participating in the washing event, the amount of fluid that was dispensed in the washing event, and the number and identity of people who have and who have not participated in the washing event.

### BRIEF DESCRIPTION OF THE DRAWINGS

FIG. 1 illustrates a floor plan of a healthcare area utilizing the hygiene monitoring system of the present disclosure;

FIG. 2 is a diagram illustrating some of the components of an exemplary embodiment of the hygiene monitoring system of the present disclosure;

FIG. 3 is a flowchart illustrating steps taken by an exemplary embodiment of the hygiene monitoring system;

FIG. 4 is a block diagram illustrating the components of the hand wash register in accordance with an exemplary embodiment of the present disclosure;

FIG. 5 is a flowchart illustrating steps taken by an exemplary embodiment of the hygiene monitoring system to identify soap dispenser use; and

FIG. 6 is a diagram illustrating use of the soap dispenser of the hygiene monitoring system of the present disclosure in use.

### DETAILED DESCRIPTION

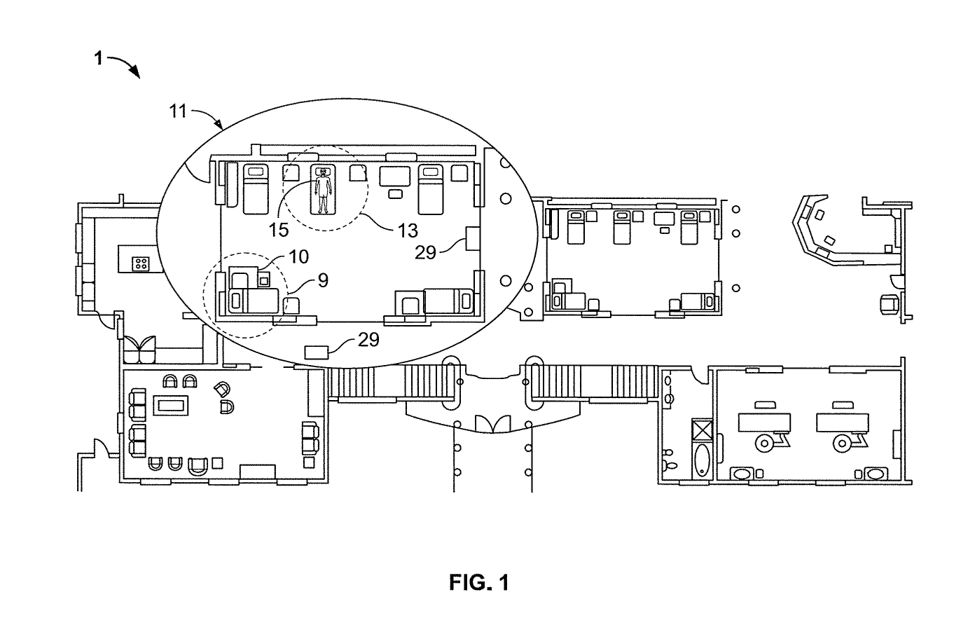

Referring to FIG. 1, a floor plan of a healthcare area 11 is shown, where a user wishes to monitor hygiene within healthcare area 11 using the hygiene monitoring system 1 of the present disclosure. Healthcare area 11 includes a hygiene zone 9 and a patient zone 13. The present disclosure refers to a healthcare area for ease of discussion. But the present disclosure is not limited to use in just the healthcare industry. The system 1 can be used in, for example, in any facility that desires to monitor and improve hygiene including, for example, restaurants, military facilities and offices. The healthcare area 11 can be identified by a virtual floor plan of a given area and includes a soap dispenser 10 within it, for example, in an office bathroom or a patient room. The embodiments described herein will use a healthcare facility scenario. However, as mentioned above, system 1 is not limited to only a healthcare scenario.

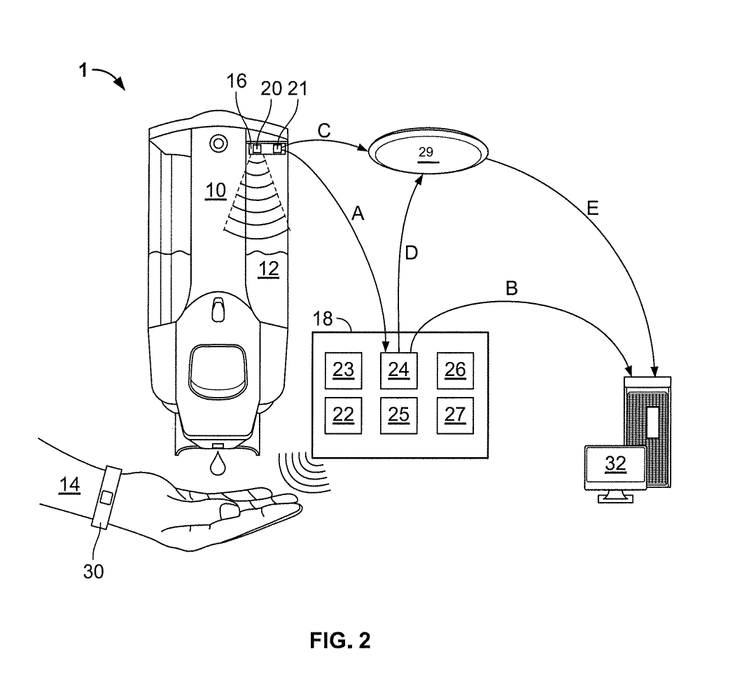

On the floor plan, indicia as defined by a geo-fence (for example, a rectangle or circular area) to define a predetermined distance can be drawn around each of the soap dispensers 10 (for example 2 meters). The system 1 can then monitor any tracking tag 30 (shown in FIG. 2) within the hygiene zone 9. Hygiene zone 9 can be the area within a predetermined distance of soap dispenser 10. The system 1 may further include a patient zone 13. The patient zone 13 is a dynamic virtual perimeter around the patient 15. As the patient moves, patient zone 13 moves accordingly. Subsequently, software rules can be defined that activates the hygiene monitoring system 1 and determine that a hand washing event is required, for example, when a tracking tag 30 enters or exits any of the healthcare area 11 or patient zone 13. The rules governing when a hand wash event is required can vary. In some embodiments of the system 1, the healthcare area 11 can be defined by a geo-fence. Various location sensors 29, discussed in further detail below, can track the precise location of any person or patient 15 within healthcare area 11, including determining when they enter or exit healthcare area 11.

It is noted that the liquid soap dispenser 10 may contain soap 12, alcohol, hand sanitizer, or a combination. For brevity, throughout this disclosure, the term “soap dispenser” or “liquid soap dispenser” 10 shall mean a dispenser 10 capable of dispensing soap, alcohol, and/or hand sanitizer 12. Further, throughout this disclosure, the term “soap” 12 shall mean liquid soap, alcohol, or hand sanitizer.

Now referring to FIG. 2, a diagram illustrating the components of the hygiene monitoring system 1 of the present disclosure is shown. The soap dispenser 10 shown in FIG. 1 is just an example of a type of liquid soap dispenser 10 and the present disclosure is not limited to the specific soap dispenser 10 depicted in FIG. 1. The soap dispenser 10 can either be manual or an electronic dispenser of the type that releases soap 12 when the user or person 14 waives their hand near a sensor or touches or otherwise interfaces with the sensor. In some embodiments, the soap dispenser 10 can include visual communication indicia or devices. For example, the soap dispenser 10 may include a shrink wrap adhesive that covers the dispenser 10 which allows signaling sounds, colors, texts, and/or images to provide instructions, campaigns, warnings, advertisements, etc., to the person. In other embodiments, the soap dispenser 10 may include a device-integrated Light Emitting Diode (LED) screen (not shown) to allow for interactive communication with a person 14.

In one embodiment of the present disclosure, the hygiene monitoring system 1 contains location sensors 29 within healthcare area 11, which can be based on any location-generating technology (e.g., a BLE, wifi, ultrawideband, and any other XY or XYZ coordinate system), and which are able to determine the precise location of persons 14 and patients 15 within healthcare area 11 by communicating with tracking tags. The location sensors 29 are further capable of receiving information from various components including tracking tag 30, SLM 16 and HWR 18, and capable of transmitting this information to computing device 32. The computing device 32 can be a local computer, a remote access server, or any suitable platform for monitoring, managing, processing and storing information. The user of hygiene monitoring system 1 can input pre-defined rules into the computing device 32 as to whether a hand washing event is required based upon, for example, the location of person 14 within the healthcare area 11, the hygiene zone 9, or the patient zone 13, the proximity between person 14 and a patient 15, etc. Any pre-defined rules determining when a hand wash event should occur, may be implemented.

In another embodiment of the present disclosure, the hygiene monitoring system 1 includes a Soap Level Meter (SLM) 16 and Hand Wash Register (HWR) 18. Both components can work separately or in conjunction with each other. The components can work, for example, as stand-alone devices or may communicate with each other using any type of communication protocol.

An exemplary embodiment of the HWR 18 including a sensor 22, a detection module 23, a communication module 24, a processor 25, a memory 26, and circuitry 27, is depicted in FIG. 2.

The sensor 22 detects when a person 14 has placed their hands underneath the soap dispenser 10. The sensor 22 may be, for example, a low power infrared (IR) or capacitive sensing proximity sensor. The HWR 18 may store this information in, for example, memory 26 or in a computing device 32, as discussed below.

The detection module 23 detects the identity of the person 14 or patient 15 by reading the unique identification information contained within a tracking tag 30. The tracking tags 30 can be worn by persons 14 and patients 15. The detection module 23 may be any ultrahigh frequency (UHF) radio frequency identification (RFID) detection technology (e.g., Bluetooth Low Energy (BLE)). The information collected by the detection module 23 is sent to the location sensor 29 or the computing device 32.

An exemplary embodiment of the SLM 16 includes a measuring module 20 and a communication interface 21. The measuring module 20 of the SLM 16 measures the level of soap 12 contained in a soap dispenser 10. The communication interface 21 of the SLM 16 is configured to send information that includes the level of soap 12 to the HWR 18 or the location sensor 29.

The measuring module 20 may take soap level measurements by any standard level measuring techniques. For example, in one non-limiting embodiment, ultrasonic level measurement is used. Ultrasonic is the sound above human hearing range. Humans can generally hear a maximum frequency of 20 KHz. Ultrasonic frequencies are above 20 KHz. Ultrasonic waves may be used to measure level of liquids and solid objects in industries. Ultrasonic level measurement is a contactless principle and most suitable for level measurements of hot, corrosive, and boiling liquids. Ultrasonic pulse signals are sent out by the measuring module 20 and may be targeted towards an object or liquid—in this case, the soap 12. The pulse signals are reflected by the soap 12 and an echo returns to the measuring module 20. The time traveled by the ultrasonic pulse is calculated by the measuring module 20, and the distance of the object is determined.

The SLM 16 can, depending on its position, measure the level of fluid (if mounted in the bottom inside the dispenser 10) or level of air (if mounted on the top inside of the dispenser 10). The SLM 16 may be mounted anywhere near, on, or within soap dispenser 10 and the present disclosure is not limiting in this regard.

In one embodiment, the SLM 16 is included in the soap dispenser 10, and is capable of determining the level of the soap 12 in the soap dispenser 10. The SLM 16 monitors the level of soap 12 in soap dispenser 10 by taking periodic measurements of the level of soap 12. The periodic measurements may be triggered at set time intervals or times of day; or the SLM 16 may take a measurement in response to an external event such as a person 14 using the soap dispenser 10 or the HWR 18 or the computing device 32 requesting a measurement. The SLM 16 may transmit this information to either the location sensor 29 (Arrow C) or the HWR 18 (Arrow A). In another embodiment, the SLM 16 may be separate from but in communication with the soap dispenser 10.

The communication module 24 is configured to receive information from the sensor 22, the detection module 23, the SLM 16 (e.g., level of soap 12), and the computing device 32. The communication module 24 may be, for example, a short-range radio communication device such as Bluetooth or similar technology, a general packet radio service, or wifi controller. The communication module 24 is further configured to transmit that information either to the location sensor 29 (Arrow D) and then to a computing device 32 (Arrow E); or directly to the computing device 32 (Arrow B).

Processing circuitry 27 may include integrated circuitry for processing and/or control, e.g., one or more processors 25 and/or processor cores and/or FPGAs (Field Programmable Gate Array) and/or ASICs (Application Specific Integrated Circuitry). The processing circuitry 27 may include and/or be connected to and/or be configured for accessing (e.g., writing to and/or reading from) the memory 26, which may comprise any kind of volatile and/or non-volatile memory, e.g., cache and/or buffer memory and/or RAM (Random Access Memory) and/or ROM (Read-Only Memory) and/or optical memory and/or EPROM (Erasable Programmable Read-Only Memory). Such memory 26 may be configured to store code executable by processing circuitry 27 and/or other data, e.g., data pertaining to communication, e.g., configuration and/or address data of nodes, etc. The processing circuitry 27 may be configured to control any of the methods described herein and/or to cause such methods to be performed, e.g., by the processor 25. Corresponding instructions may be stored in the memory 26, which may be readable and/or readably connected to the processing circuitry 27. In other words, the processing circuitry 27 may include a controller, which may comprise a microprocessor and/or microcontroller and/or FPGA (Field-Programmable Gate Array) device and/or ASIC (Application Specific Integrated Circuit) device. It may be considered that the processing circuitry includes or may be connected or connectable to memory 26, which may be configured to be accessible for reading and/or writing by the controller and/or processing circuitry 27.

The present disclosure is not limited to the particular hardware depicted in FIG. 2, and therefore any compatible hardware may be used.

A scenario of the exemplary hygiene system 1 in use is as follows: a person 14 enters a healthcare area 11. The person's location within healthcare area 11 is determined by location sensors 29. Having entered healthcare area 11, the backend computing device 32 processes and determines, based upon the customizable, predefined software rules entered into the frontend user interface of the system by the facility, that he or she must therefore wash his/her hands in accordance with the hand hygiene policy of the facility. As described above, this is just one way to indicate that a hand washing event is required. The occurrence or compliance with a hand washing event is verified by one or more of the following mechanisms. First, detection module 23 determines if the person 14 has a tracking tag 30 and is within proximity of the hygiene zone 9, i.e., within proximity to dispenser 10. Since tracking tag 30 is coded with information identifying person 14, detection module 23 is able to determine the identity of the wearer of the tracking tag 30 and therefore determine the identity of the person 14 within proximity of soap dispenser 10. When the person 14 is detected within the hygiene zone 9, a hand washing event may be inferred.

Second, if sensor 22 detects when a person 14 has placed his or her hands underneath the soap dispenser 10, it can be inferred that person 14 is using soap dispenser 10, has washed their hands, and has complied with the requisite hygiene protocol.

Third, the communication module 24 of the HWR 18, the computing device 32, or some other source, transmits a communication signal to communication interface 21 of the SLM 16, instructing the measuring module 20 of the SLM 16 to take a soap level measurement. In response, communication interface 21 of SLM 16 transmits information including the level measurement of soap 12 in dispenser 10 to the communication module 24 of HWR 18 (Arrow A) or to the computing device 32. HWR 18 compares the most recent soap level measurement (i.e., the measurement taken after person 14 has exited) to a preceding soap level measurement stored in the memory 26 and/or in the computing device 29. If the soap level measurement has decreased, then the system 1 has determined that the person 14 has used soap dispenser 10 and washed their hands in compliance with the hygiene protocol.

In other embodiments, SLM 16 takes soap level measurements at periodic intervals. Thus, the present disclosure advantageously provides multiple methods in which to determine if a hand wash event has occurred. One, two, or all three of these methods may be used depending upon the requirements of the user of hygiene monitoring system 1.

In one embodiment, information relating to the identity of the person 14 and their compliance (or non-compliance) with the hygiene protocol may be sent to the computing device 32 by the communication module 24 of HWR 18 (Arrow B).

Although not shown in FIG. 2, in some embodiments, the HWR 18 can be housed in a small receptacle and mounted inside or to a surface of the soap dispenser 10. In another embodiment, the HWR 18 is not mounted to the soap dispenser 10 but is placed nearby and in communication with the soap dispenser 10.

The SLM 16 may send soap level information directly to the location sensor 29 (Arrow C). The HWR 18 may also send information about the identity of person 14, e.g., that person's compliance (or non-compliance) with hygiene protocol, and the length of the washing event to the location sensor 29 (Arrow D). The location sensor 29 can send the information to the computing device 32 (Arrow E).

In another embodiment, the communication interface 21 of the SLM 16 can transmit the information relating to soap level “on demand” by phones, tablets, or any other electronic communication devices, rather than upon an instruction signal from HWM 18 or the computing device 32. In one embodiment, the necessary settings and calibration can be done by Android/iOS applications, or any other types of smartphone applications, or from any backend services, if available.

In some embodiments, the SLM 16 is powered by a battery, for example, a CR2032 Lithium 3 volt battery. The present disclosure is not limited to a particular type of power source or a particular power source capacity.

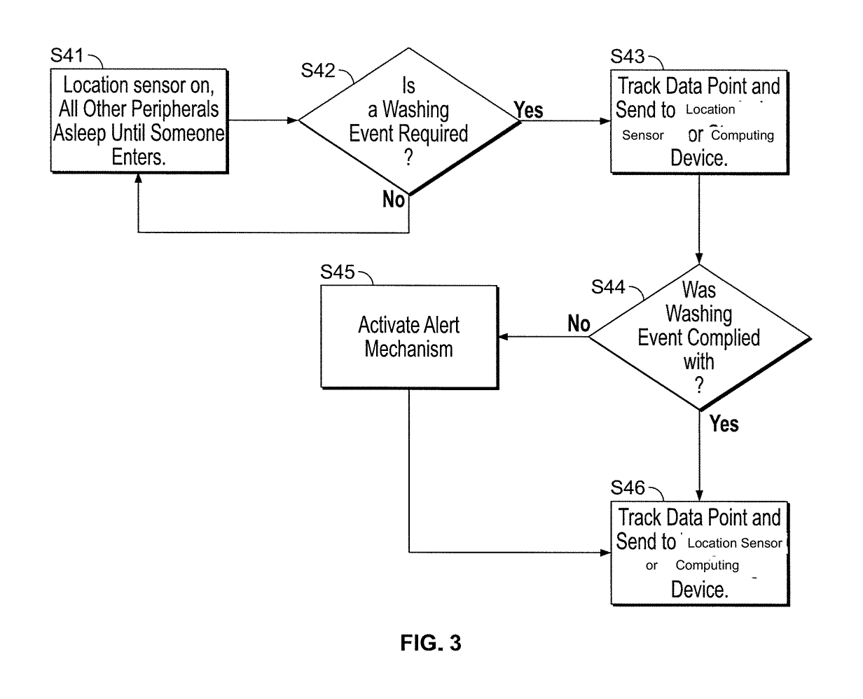

In FIG. 3, a flowchart of an exemplary embodiment of the hygiene monitoring system 1 is shown. At step S41, the hygiene monitoring system 1 is in standby mode with all peripherals sleeping, except for the location sensors 29. These sensors are constantly detecting the presence and exact location of people within healthcare area 11. Then, at step S42, the computing device 32 determines if a hand washing event is required based on the customized rules. The requirement of a hand washing event is customized by each facility. Examples of hand washing event requirements may include, for example: a person 14 enters a healthcare area 11 or a patient zone 13 or a person 14 exits a patient zone 13 or healthcare area 11. If a hand washing event is not required, the hygiene monitoring system 1 stays in standby mode S41. If a hand washing event is required, data points are transmitted from tracking tag 30 to location sensor 29 to the computing device 32 at step S43. Then, at step S44, the hygiene monitoring system 1 determines whether a hand washing event has been completed. As described above, hygiene monitoring system 1 may use three ways to track whether a hand washing event occurred: first, if the sensor 22 detects hands underneath the soap dispenser 10; second, if the detection module 23 detects a person's 14 tracking tag 30 within the hygiene zone 9; third, if the SLM 16 detects a change in the level of soap. If hand washing has occurred, the data is tracked and sent to the location sensor 29 and/or the computing device 32 at step S46. If the hand washing event has not occurred, in one embodiment, an alert mechanism 38 is activated at step S45. The data is then tracked and sent to location sensor 29 and/or computing device 32 at step S46.

The alert mechanism 38 may be included on the tracking tag 30, on the HWR 18, or as a separate unit. The alert mechanism 38 may be activated at any one or more times that a required hand washing event is not complied with. This, like the rules determining hand washing requirements, is determined by the facility. The invention is not limited to a specific element that triggers the alert. It could be computing device 32 and software therein, or HWR 18, or any other element of system 1. The alert mechanism 38 is not limited in the type of alert that can be produced. For example, it could be a sound or a visual alert such as a flashing light or a tactile alert such as a buzzer. In one embodiment, tag 30 includes an LED display that can flash or change color (for example, from green to red) and/or a buzzer than can be triggered if a required hand wash event has not occurred.

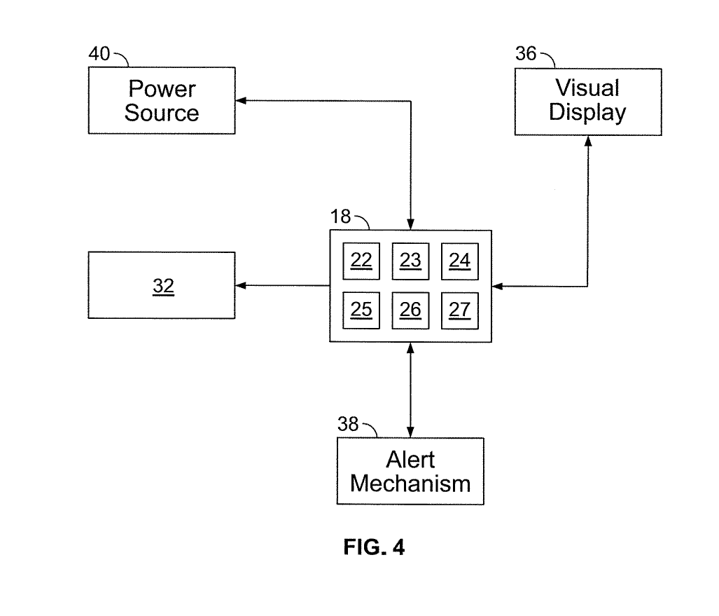

Referring to FIG. 4, a block diagram of an exemplary embodiment of the HWR 18 is shown including additional components. Alternative embodiments of the HWR 18 can include one or more of an alert mechanism 38 (e.g., a speaker, a buzzer, a flashing light, or LED), a power source 40 (e.g., a battery or AC power), and a visual display 36 (e.g., an LCD screen). These additional components may be included in HWR 18 as a single unit or be separate from HWR 18 but in communication with the HWR 18.

In embodiments with a visual display 36, the visual display 36 is part of or in communication with the HWR 18. Visual display 36 may be configured, via processor 25, to display such information as, but not limited to, the identity of the person 14, the level of soap in dispenser 10, and/or an alert message. As described above, information regarding the hand washing event (or lack thereof), and the identity of the person 14 who has or has not washed their hands, may be transmitted to the location sensor 29 and/or the computing device 32.

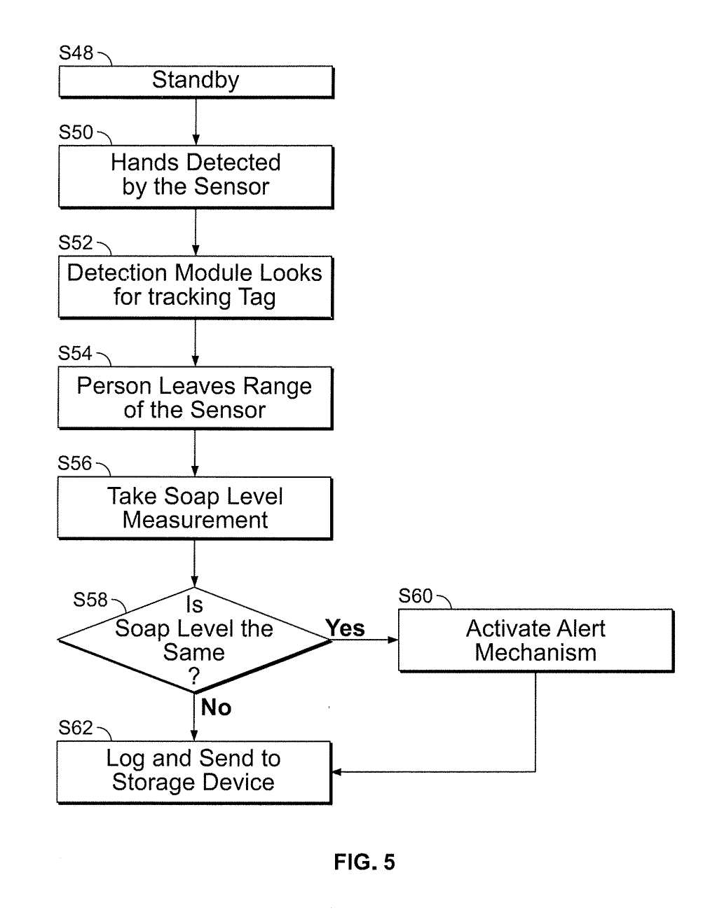

In FIG. 5, a flow chart of exemplary steps taken by an exemplary embodiment of the hygiene monitoring system 1. At step S48, all peripherals are sleeping but the sensor 22 of HWR 18 is in active mode waiting to detect the presences of a person's 14 hands. At step S50, hands are placed underneath or otherwise near dispenser 10 and are thus detected by the sensor 22. At step S52, the detection module 23 looks for a tracking tag 30 worn by person 14. The presence or absence of a tracking tag 30 determines if an additional data point relating to identity of person 14 will be stored. At step S54, the person 14 has walked away from the sensor 22 (e.g., outside of a predetermined range). At step S56, the communication module 24 or the computing device 32 transmits a signal to the communication interface 21 of the SLM 16 instructing the SLM 16 to take a soap level measurement. That measurement is sent back to the HWR 18, the location sensor 29, and/or the computing device 32. At step S58, computing device 32 compares the new measurement to the previous measurement that is stored in the memory 26 or the computing device 32. If the soap level measurement has decreased, indicating that the soap dispenser 10 has been used by the person 14, all relevant data points are sent to or stored by the computing device 32, at step S62, either directly or via location sensor 29. If the soap level has not changed, in one embodiment, the alert mechanism 38 is activated, at step S60. In one embodiment, relevant data points are sent to the computing device 32 at step S62.

It should be noted that one, two or three of the mechanisms described above for determining if a hand wash event has occurred may be implemented. Thus, it may not be required or desired to determine if SLM 16 has detected a change in soap level. The occurrence of the hand wash event may be determined by sensor 22 and/or detection module 23 in the aforementioned manner.

Regardless of whether a tracking tag 30 is worn by person 14, data collected by SLM 16 and HWR 18 can include, e.g., soap level, room number, and the time of day that the washing event occurred. If the tracking tag 30 is in use, the system 1 collects additional data including the identity of person 14.

Optionally, a patient 15 within healthcare area 11 may also wear a tracking tag 30. At step S52, the detection module 23 would then look for and log data relating to both the person 14 and the patient 15 by sensing both tracking tags 30, i.e., the tracking tag 30 on person 14 and tracking tag 30 on patient 15.

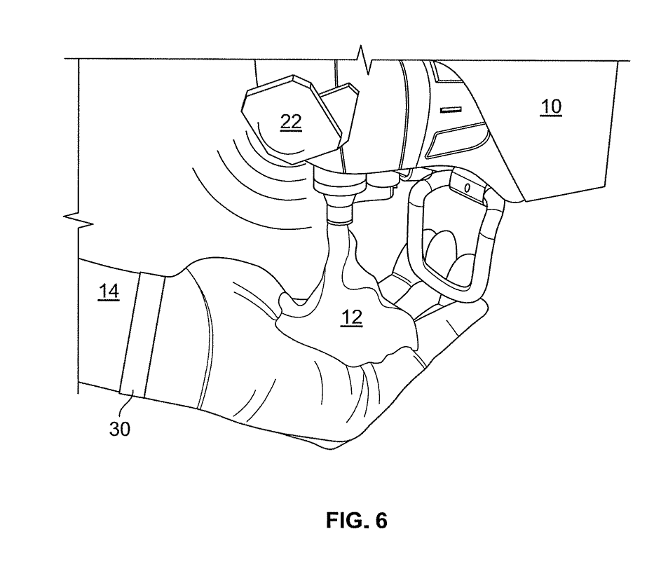

FIG. 6 illustrates an example of the hygiene monitoring system 1 of the present disclosure in use. In this example, person 14, wearing a tracking tag 30 as a bracelet with an active UHF RFID tag uses soap dispenser 10 with sensor 22 mounted on it. As described above, the HWR 18 detects the identity of the person 14 via the tracking tag 30. The SLM 16 and HWR 18 are used together to gather information regarding the requirement of a washing event, whether the washing event was complied with, the identity of the person 14 using the soap dispenser 10, the time the soap dispenser 10 was used, and send that information to a location sensor 29 or computing device 32 for analysis and reporting.

In another embodiment, the tracking tag 30 will be an RFID identification badge or card (not shown). The soap dispenser 10 can be equipped with a card/badge reader. The person 14 can insert or touch their ID card to the reader and thus identify themselves.

Now, it will be helpful to understanding the hygiene monitoring system 1 by way of describing exemplary use-case scenarios.

Exemplary Use Case 1:
1. Person 14 enters the healthcare area 11 which is detected by location sensors 29.
2. Hygiene monitoring system 1 is activated.
3. Location sensors 29 detect the patient 15 is present.
4. Data point is recorded (identity of person 14 via tracking tag 30, time, room #, location—entrance of healthcare area 11, hands not washed, patient 15 present, no alert).
5. Person 14 washes hands determined by change in soap level in dispenser 10, detection of person 14 within hygiene zone 9 by detection module 23, and/or detection of hands by sensor 22.
6. Data point is recorded (identity of person 14 via tracking tag 30, time, room #, location—hygiene zone 9, hands washed, patient 15 present, no alert).
7. Person 14 moves back into healthcare area 11 as determined by location sensors 29.
8. Data point is recorded (identity of person 14 via tracking tag 30, time, room #, location—healthcare area 11, hands washed, patient 15 present, no alert).
9. Person 14 moves into patient zone 13.
10. Data point is recorded (identity of person 14 via tracking tag 30, time, room #, location—patient zone 13, hands washed, patient 15 present, no alert).
11. Person 14 moves back into healthcare area 11 which is detected by location sensors 29.
12. Data point is recorded (identity of person 14 via tracking tag 30, time, room #, location—healthcare area 11, hands not washed, patient 15 present, no alert).
13. Person 14 washes hands before exiting healthcare area 11 determined by change in soap level in dispenser 10, detection of person 14 within hygiene zone 9 by detection module 23, and/or detection of hands by sensor 22.
14. Data point is recorded (identity of person 14 via tracking tag 30, time, room #, location—hygiene zone 9, hands washed, patient 15 present, no alert).
15. Person 14 moves back into healthcare area 11.
16. Data point is recorded (identity of person 14 via tracking tag 30, time, room #, location—healthcare area 11, hands washed, patient 15 present, no alert).
17. Person 14 exits healthcare area 11.
18. Data point is recorded (identity of person 14 via tracking tag 30, time, room #, location—exit of healthcare area 11, hands washed, patient 15 present, no alert).
19. System tracking for person 14 is deactivated.

Exemplary Use Case 2:
1. Person 14 enters the healthcare area 11 which is detected by location sensors 29.
2. Hygiene monitoring system 1 is activated.
3. Location sensors 29 determine patient 15 is present.
4. Data point is recorded (identity of person 14 via tracking tag 30, time, room #, location—entrance of healthcare area 11, hands not washed, patient 15 present, no alert).
5. Person 14 washes hands determined by change in soap level in dispenser 10, detection of person 14 within hygiene zone 9 by detection module 23, and/or detection of hands by sensor 22.
6. Data point is recorded (identity of person 14 via tracking tag 30, time, room #, location—hygiene zone 9, hands washed, patient 15 present, no alert).
7. Person 14 moves back into healthcare area 11.
8. Data point is recorded (identity of person 14 via tracking tag 30, time, room #, location—healthcare area 11, hands washed, patient 15 present, no alert).
9. Person 14 moves into patient zone 13.
10. Data point is recorded (identity of person 14 via tracking tag 30, time, room #, location—patient zone 13, hands washed, patient 15 present, no alert).
11. Person 14 back into moves back healthcare area 11.
12. Data point is recorded (identity of person 14 via tracking tag 30, time, room #, location—healthcare area 11, hands not washed, patient 15 present, no alert).
13. Person 14 does not wash hands before exiting healthcare areas 11 determined by no change in soap level in dispenser 10, no detection of person 14 within hygiene zone 9 by detection module 23, and/or no detection of hands by sensor 22.
14. Person 14 exits healthcare area 11.
15. Data point is recorded (identity of person 14 via tracking tag 30, time, room #, location—exit of healthcare area 11, hands not washed, patient 15 present, no alert).
16. System tracking for person 14 is deactivated.

Exemplary Use Case 3:
1. Person 14 enters the healthcare area 11 which is detected by location sensors 29.
2. Hygiene monitoring system 1 is activated for person 14.
3. Location sensors 29 detect the patient 15 is present.
4. Data point is recorded (identity of person 14 via tracking tag 30, time, room #, location—entrance of healthcare area 11, hands not washed, patient 15 present, no alert).
5. Person 14 washes hands determined by change in soap level in dispenser 10, detection of person 14 within hygiene zone 9 by detection module 23, and/or detection of hands by sensor 22.
6. Data point is recorded (identity of person 14 via tracking tag 30, time, room #, location—hygiene zone 9, hands washed, patient 15 present, no alert).
7. Person 14 moves back into healthcare area 11.
8. Data point is recorded (identity of person 14 via tracking tag 30, time, room #, location—healthcare area 11, hands washed, patient 15 present, no alert).
9. Person 14 washes hands before exiting healthcare area 11 determined by change in soap level in dispenser 10, detection of person 14 within hygiene zone 9 by detection module 23, and/or detection of hands by sensor 22.
10. Data point is recorded (identity of person 14 via tracking tag 30, time, room #, location—hygiene zone 9, hands washed, patient 15 present, no alert).
11. Person 14 moves back into healthcare area 11.
12. Data point is recorded (identity of person 14 via tracking tag 30, time, room #, location—healthcare area 11, hands washed, patient 15 present, no alert).
13. Person 14 exists healthcare area 11.
14. Data point is recorded (identity of person 14 via tracking tag 30, time, room #, location—exit of healthcare area 11, hands washed, patient 15 present, no alert).
15. System tracking for person 14 is deactivated.

Exemplary Use Case 4:
1. Person 14 enters the healthcare area 11 which is detected by location sensors 29.
2. Hygiene monitoring system 1 is activated for person 14.
3. Location sensors 29 detect the patient 15 is present.
4. Data point is recorded (identity of person 14 via tracking tag 30, time, room #, location—entrance of healthcare area 11, hands not washed, patient 15 present, no alert).
5. Person 14 washes hands determined by change in soap level in dispenser 10, detection of person 14 within hygiene zone 9 by detection module 23, and/or detection of hands by sensor 22.
6. Data point is recorded (identity of person 14 via tracking tag 30, time, room #, location—hygiene zone 9, hands washed, patient 15 present, no alert).
7. Person 14 moves back into healthcare area 11.
8. Data point is recorded (identity of person 14 via tracking tag 30, time, room #, location—healthcare area 11, hands washed, patient 15 present, no alert).
9. Person 14 does not wash hands before exiting the healthcare area 11 determined by no change in soap level in dispenser 10, no detection of person 14 within hygiene zone 9 by detection module 23, and/or no detection of hands by sensor 22.
10. Person 14 exits healthcare area 11.
11. Data point is recorded (identity of person 14 via tracking tag 30, time, room #, location—exit of healthcare area 11, hands not washed, patient 15 present, no alert).
12. System tracking for person 14 is deactivated.

Exemplary Use Case 5:
1. Person 14 enters the healthcare area 11 which is detected by location sensors 29.
2. Hygiene monitoring system 1 is activated for person 14.
3. Location sensors 29 detect the absence of patient 15.
4. Data point is recorded (identity of person 14 via tracking tag 30, time, room #, location—entrance to healthcare area 11, hands not washed, patient 15 not present, no alert).
5. Person 14 washes hands determined by change in soap level in dispenser 10, detection of person 14 within hygiene zone 9 by detection module 23, and/or detection of hands by sensor 22.
6. Data point is recorded (identity of person 14 via tracking tag 30, time, room #, location—hygiene zone 9, hands washed, patient 15 not present, no alert).
7. Person 14 moves back into healthcare area 11.
8. Data point is recorded (identity of person 14 via tracking tag 30, time, room #, location—healthcare area 11, hands washed, patient 15 not present, no alert).
9. Person 14 washes hands before exiting healthcare area 11 determined by change in soap level in dispenser 10, detection of person 14 within hygiene zone 9 by detection module 23, and/or detection of hands by sensor 22.
10. Data point is recorded (identity of person 14 via tracking tag 30, time, room #, location—hygiene zone 9, hands washed, patient 15 not present, no alert).
11. Person 14 moves back into healthcare area 11.
12. Data point is recorded (identity of person 14 via tracking tag 30, time, room #, location—healthcare area 11, hands washed, patient 15 not present, no alert).
13. Person 14 exits healthcare area 11.
14. Data point is recorded (identity of person 14 via tracking tag 30, time, room #, location—exit of healthcare area 11, hands washed, patient 15 not present, no alert).
15. System tracking for person 14 is deactivated.

Exemplary Use Case 6:
1. Person 14 enters the healthcare area 11 which is detected by location sensors 29.
2. Hygiene monitoring system 1 is activated for person 14.
3. Location sensors 29 detect absence of patient 15.
4. Data point is recorded (identity of person 14 via tracking tag 30, time, room #, location—entrance to healthcare area 11, hands not washed, patient 15 not present, no alert).
5. Person 14 washes hands determined by change in soap level in dispenser 10, detection of person 14 within hygiene zone 9 by detection module 23, and/or detection of hands by sensor 22.
6. Data point is recorded (identity of person 14 via tracking tag 30, time, room #, location—hygiene zone 9, hands washed, patient 15 not present, no alert).
7. Person 14 moves back into healthcare area 11.
8. Data point is recorded (identity of person 14 via tracking tag 30, time, room #, location—healthcare area 11, hands washed, patient 15 not present, no alert).
9. Person 14 does not wash hands before exiting the healthcare area 11 determined by no change in soap level in dispenser 10, no detection of person 14 within hygiene zone 9 by detection module 23, and/or no detection of hands by sensor 22.
10. Person 14 exits healthcare area 11 which is detected by location sensors 29.
11. Data point is recorded (identity of person 14 via tracking tag 30, time, room #, location—exit of healthcare area 11, hands not washed, patient 15 not present, no alert).
12. System tracking for person 14 is deactivated.

Exemplary Use Case 7:
1. Person 14 enters the healthcare area 11 which is detected by location sensors 29.
2. Hygiene monitoring system 1 is activated for person 14.
3. Location sensors 29 detects the patient 15 is present.
4. Data point is recorded (identity of person 14 via tracking tag 30, time, room #, location—entrance to healthcare area 11, hands not washed, patient 15 present, no alert).
5. Person 14 does not wash hands determined by no change in soap level in dispenser 10, no detection of person 14 within hygiene zone 9 by detection module 23, and/or no detection of hands by sensor 22.
6. Person 14 moves into patient zone 13.
7. Alert mechanism 38 is activated.
8. Data point is recorded (identity of person 14 via tracking tag 30, time, room #, location—patient zone 13, hands not washed, patient 15 present, alert).
9. Person 14 moves back into healthcare area 11.
10. Data point is recorded (identity of person 14 via tracking tag 30, time, room #, location—healthcare area 11, hands not washed, patient 15 present, no alert).
11. Person 14 washes hands determined by change in soap level in dispenser 10, detection of person 14 within hygiene zone 9 by detection module 23, and/or detection of hands by sensor 22.
12. Data point is recorded (identity of person 14 via tracking tag 30, time, room #, location—hygiene zone 9, hands washed, patient 15 present, no alert).
13. Person 14 moves back into healthcare area 11.
14. Data point is recorded (identity of person 14 via tracking tag 30, time, room #, location—healthcare area 11, hands washed, patient 15 present, no alert).
15. Person 14 moves back into patient zone 13.
16. Data point is recorded (identity of person 14 via tracking tag 30, time, room #, location—patient zone 13, hands washed, patient 15 present, no alert).
17. Person 14 moves back into healthcare area 11.
18. Data point is recorded (identity of person 14 via tracking tag 30, time, room #, location—healthcare area 11, hands not washed, patient 15 present, no alert).
19. Person 14 washes hands before exiting healthcare area 11 determined by change in soap level in dispenser 10, detection of person 14 within hygiene zone 9 by detection module 23, and/or detection of hands by sensor 22.
20. Data point is recorded (identity of person 14 via tracking tag 30, time, room #, location—hygiene zone 9, hands washed, patient 15 present, no alert).
21. Person 14 exits healthcare area 11.
22. Data point is recorded (identity of person 14 via tracking tag 30, time, room #, location—exit of healthcare area 11, hands washed, patient 15 present, no alert).
23. System tracking for person 14 is deactivated.

Exemplary Use Case 8:
1. Person 14 enters the healthcare area 11 which is detected by location sensors 29.
2. Hygiene monitoring system 1 is activated for person 14.
3. Location sensors 29 detect the patient 15 is present.
4. Data point is recorded (identity of person 14 via tracking tag 30, time, room #, location—entrance to healthcare area 11, hands not washed, patient 15 present, no alert).
5. Person 14 does not wash hands determined by no change in soap level in dispenser 10, no detection of person 14 within hygiene zone 9 by detection module 23, and/or no detection of hands by sensor 22.
6. Person 14 moves into patient zone 13.
7. Alert mechanism 38 is activated.
8. Data point is recorded (identity of person 14 via tracking tag 30, time, room #, location—patient zone 13, hands not washed, patient 15 present, alert).
9. Person 14 moves back into healthcare area 11.
10. Data point is recorded (identity of person 14 via tracking tag 30, time, room #, location—healthcare area 11, hands not washed, patient 15 present, no alert).
11. Person 14 washes hands determined by change in soap level in dispenser 10, detection of person 14 within hygiene zone 9 by detection module 23, and/or detection of hands by sensor 22.
12. Data point is recorded (identity of person 14 via tracking tag 30, time, room #, location—hygiene zone 9, hands washed, patient 15 present, no alert).
13. Person 14 moves into healthcare area 11.
14. Data point is recorded (identity of person 14 via tracking tag 30, time, room #, location—healthcare area 11, hands washed, patient 15 present, no alert).
15. Person 14 moves back into patient zone 13.
16. Data point is recorded (identity of person 14 via tracking tag 30, time, room #, location—patient zone 13, hands washed, patient 15 present, no alert).
17. Person 14 moves back into healthcare area 11.
18. Data point is recorded (identity of person 14 via tracking tag 30, time, room #, location—healthcare area 11, hands not washed, patient 15 present, no alert).
19. Person 14 does not wash hands before exiting healthcare area 11 determined by no change in soap level in dispenser 10, no detection of person 14 within hygiene zone 9 by detection module 23, and/or no detection of hands by sensor 22.
20. Person 14 exits healthcare area 11.
21. Data point is recorded (identity of person 14 via tracking tag 30, time, room #, location—exit of healthcare area 11, hands not washed, patient 15 present, no alert).
22. System tracking for person 14 is deactivated.

Exemplary Use Case 9:
1. Person 14 enters the healthcare area 11 which is detected by location sensors 29.
2. Hygiene monitoring system 1 is activated for person 14.
3. Location sensors 29 detect the patient 15 is present.
4. Data point is recorded (identity of person 14 via tracking tag 30, time, room #, location—entrance to healthcare area 11, hands not washed, patient 15 present, no alert).
5. Person 14 does not wash hands determined by no change in soap level in dispenser 10, no detection of person 14 within hygiene zone 9 by detection module 23, and/or no detection of hands by sensor 22.
6. Person 14 moves into patient zone 13.
7. Alert mechanism 38 is activated.
8. Data point is recorded (identity of person 14 via tracking tag 30, time, room #, location—patient zone 13, hands not washed, patient 15 present, alert).
9. Person 14 remains within patient zone 13 and does not wash hands determined by no change in soap level in dispenser 10, no detection of person 14 within hygiene zone 9 by detection module 23, and/or no detection of hands by sensor 22.
10. Person 14 moves back into healthcare area 11.
11. Data point is recorded (identity of person 14 via tracking tag 30, time, room #, location—healthcare area 11, hands not washed, patient 15 present, no alert).
12. Person 14 washes hands before exiting healthcare area 11 as determined by change in soap level in dispenser 10, detection of person 14 within hygiene zone 9 by detection module 23, and/or detection of hands by sensor 22
13. Data point is recorded (identity of person 14 via tracking tag 30, time, room #, location—hygiene zone 9, hands washed, patient 15 present, no alert).
14. Person 14 moves back into healthcare area 11.
15. Data point is recorded (identity of person 14 via tracking tag 30, time, room #, location—healthcare area 11, hands washed, patient 15 present, no alert).
16. Person 14 exits healthcare area 11.
17. Data point is recorded (identity of person 14 via tracking tag 30, time, room #, location—exit of healthcare area 11, hands washed, patient 15 present, no alert).
18. System tracking for person 14 is deactivated.

Exemplary Use Case 10:
1. Person 14 enters the healthcare area 11 which is detected by location sensors 29.
2. Hygiene monitoring system 1 is activated for person 14.
3. Location sensors 29 detect the patient 15 is present.
4. Data point is recorded (identity of person 14 via tracking tag 30, time, room #, location—entrance to healthcare area 11, hands not washed, patient 15 present, no alert).
5. Person 14 does not wash hands determined by no change in soap level in dispenser 10, no detection of person 15 within hygiene zone 9 by detection module 23, and/or no detection of hands by sensor 22.
6. Person 14 moves into patient zone 13.
7. Alert mechanism 38 is activated.
8. Data point is recorded (identity of person 14 via tracking tag 30, time, room #, location—patient zone 13, hands not washed, patient 15 present, alert).
9. Person 14 remains within patient zone 13 and does not wash hands.
10. Person 14 moves back into healthcare area 11.
11. Data point is recorded (identity of person 14 via tracking tag 30, time, room #, location—healthcare area 11, hands not washed, patient 15 present, no alert).
12. Person 14 does not wash hands before exiting healthcare area 11 determined by no change in soap level in dispenser 10, no detection of person 14 within hygiene zone 9 by detection module 23, and/or no detection of hands by sensor 22.
13. Person 14 exits healthcare area 11.
14. Data point is recorded (identity of person 14 via tracking tag 30, time, room #, location—exit of healthcare area 11, hands not washed, patient 15 present, no alert).
15. System tracking for person 14 is deactivated.

Exemplary Use Case 11:
1. Person 14 enters the healthcare area 11 which is detected by location sensors 29.
2. Hygiene monitoring system 1 is activated for person 14.
3. Location sensors detect the patient 15 is present.
4. Data point is recorded (identity of person 14 via tracking tag 30, time, room #, location—entrance to healthcare area 11, hands not washed, patient 15 present, no alert).
5. Person 14 does not wash hands determined by no change in soap level in dispenser 10, no detection of person 14 within hygiene zone 9 by detection module 23, and/or no detection of hands by sensor 22.
6. Person 14 washes hands before exiting healthcare area 11 determined by change in soap level in dispenser 10, detection of person 14 within hygiene zone 9 by detection module 23, and/or detection of hands by sensor 22.
7. Data point is recorded (identity of person 14 via tracking tag 30, time, room #, location—hygiene zone 9, hands washed, patient 15 present, no alert).
8. Person 14 moves back into healthcare area 11.
9. Data point is recorded (identity of person 14 via tracking tag 30, time, room #, location—healthcare area 11, hands washed, patient 15 present, no alert).
10. Person 14 exits healthcare area 11.
11. Data point is recorded (identity of person 14 via tracking tag 30, time, room #, location—exit of healthcare area 11, hands washed, patient 15 present, no alert).
12. System tracking for person 14 is deactivated.

Exemplary Use Case 12:
1. Person 14 enters the healthcare area 11 which is detected by location sensors 29.
2. Hygiene monitoring system 1 is activated for person 14.
3. Location sensors 29 detect the patient 15 is present.
4. Data point is recorded (identity of person 14 via tracking tag 30, time, room #, location—entrance to healthcare area 11, hands not washed, patient 15 present, no alert).
5. Person 14 does not wash hands determined by no change in soap level in dispenser 10, no detection of person 14 within hygiene zone 9 by detection module 23, and/or no detection of hands by sensor 22.
6. Person 14 exits healthcare area 11.
7. Data point is recorded (identity of person 14 via tracking tag 30, time, room #, location—exit of healthcare area 11, hands not washed, patient 15 present, no alert).
8. System tracking for person 14 is deactivated.

Exemplary Use Case 13:
1. Person 14 enters the healthcare area 11 which is detected by location sensors 29.
2. Hygiene monitoring system 1 is activated for person 14.
3. Location sensors 29 detect the absence of patient 15.
4. Data point is recorded (identity of person 14 via tracking tag 30, time, room #, location—entrance to healthcare area 11, hands not washed, patient 15 not present, no alert).
5. Person 14 does not wash hands determined by no change in soap level in dispenser 10, no detection of person 14 within hygiene zone 9 by detection module 23, and/or no detection of hands by sensor 22.
6. Person 14 washes hands before exiting healthcare area 11 determined by change in soap level in dispenser 10, detection of person 14 within hygiene zone 9 by detection module 23, and/or detection of hands by sensor 22.
7. Data point is recorded (identity of person 14 via tracking tag 30, time, room #, location—hygiene zone 9, hands washed, patient 15 not present, no alert).
8. Person 14 moves back into healthcare area 11.
9. Data point is recorded (identity of person 14 via tracking tag 30, time, room #, location—healthcare area 11, hands washed, patient 15 not present, no alert).
10. Person 14 exits healthcare area.
11. Data point is recorded (identity of person 14 via tracking tag 30, time, room #, location—exit of healthcare area 11, hands washed, patient 15 not present, no alert).
12. System tracking for person 14 is deactivated.

Exemplary Use Case 14:
1. Person 14 enters the healthcare area 11 which is detected by location sensors 29.
2. Hygiene monitoring system 1 is activated for person 14.
3. Location sensors 29 detect the absence of patient 15.
4. Data point is recorded (identity of person 14 via tracking tag 30, time, room #, location—entrance to healthcare area 11, hands not washed, patient 15 not present, no alert).
5. Person 14 does not wash hands determined by no change in soap level in dispenser 10, no detection of person 14 within hygiene zone 9 by detection module 23, and/or no detection of hands by sensor 22.
6. Person 14 does not wash hands before exiting healthcare area 11 determined by no change in soap level in dispenser 10, no detection of person 14 within hygiene zone 9 by detection module 23, and/or no detection of hands by sensor 22.
7. Person 14 exits healthcare area 11.
8. Data point is recorded (identity of person 14 via tracking tag 30, time, room #, location—exit of healthcare area 11, hands not washed, patient 15 not present, no alert).
9. System tracking for person 14 is deactivated.

The preceding scenarios are not exhaustive and are only meant to provide examples of the applicability of the hygiene monitoring system 1 of the present disclosure.

In embodiments of the hygiene monitoring system 1, examples of the types of reports that may be useful in improving the hygiene of healthcare facilities include but are not limited to: compliance rate of staff having washed their hands when entering a healthcare area 11 or patient zone 13 with a patient present; compliance rate of staff having washed their hands when entering a healthcare area 11 with no patient present; compliance rate of staff having washed their hands when exiting a healthcare area 11 or patient zone 13 with a patient present; compliance rate of staff having washed their hands when exiting a healthcare area 11 or patient zone 13 with no patient present; compliance rate with an alert; traceability for a specific healthcare area 11 or patient zone 13 in which an infection has occurred, including the determination of persons who entered and exited the infectious area, where they were or went before and after being in contact with the infections area; and details of breaches in compliance.

In addition to the soap dispenser 10 embodiments discussed above, the SLM 16 component of the present disclosure can be used in different scenarios. For example, the SLM 16 may be added to a bathtub to measure water level when filling the tub. That information can be used to warn a child or parent when the water level exceeds a certain level. This information could be useful in preventing drowning accidents with children or the elderly. A temperature sensor can be added in order to extend the warning application system to cover water temperature. In another embodiment, the SLM 16 can be added to water bottles to keep track of the water level for automated replenishment signaling. In another embodiment, a monitoring and alerting system can be provided for any water container, drum, or well, in order to signal check-ups, refills, shortages etc. This can be used, for example, in remote areas where water is scarce (e.g., desert/dry areas of Africa). Sensors such as, for example, temperature, bacteria, battery/solar powered, or Global System for Mobile communication (GSM)-based sensors can be added to the system. The present disclosure can also be applied to food containers. For example, inventory management can be done on sauces, soups etc. This solution, which could also be combined with temperature sensors, could save on human labor in catering scenarios.

As will be appreciated by one of skill in the art, the concepts described herein may be embodied as a method, data processing system, and/or computer program product. Accordingly, the concepts described herein may take the form of an entirely hardware embodiment, an entirely software embodiment or an embodiment combining software and hardware aspects all generally referred to herein as a “circuit” or “module.” Furthermore, the disclosure may take the form of a computer program product on a tangible computer usable storage medium having computer program code embodied in the medium that can be executed by a computer. Any suitable tangible computer readable medium may be utilized including hard disks, CD-ROMs, electronic storage devices, optical storage devices, or magnetic storage devices.

Some embodiments are described herein with reference to flowchart illustrations and/or block diagrams of methods, systems and computer program products. It will be understood that each block of the flowchart illustrations and/or block diagrams, and combinations of blocks in the flowchart illustrations and/or block diagrams, can be implemented by computer program instructions. These computer program instructions may be provided to a processor of a general purpose computer (to thereby create a special purpose computer), special purpose computer, or other programmable data processing apparatus to produce a machine, such that the instructions, which execute via the processor of the computer or other programmable data processing apparatus, create means for implementing the functions/acts specified in the flowchart and/or block diagram block or blocks.

These computer program instructions may also be stored in a computer readable memory or storage medium that can direct a computer or other programmable data processing apparatus to function in a particular manner, such that the instructions stored in the computer readable memory produce an article of manufacture including instruction means which implement the function/act specified in the flowchart and/or block diagram block or blocks.

The computer program instructions may also be loaded onto a computer or other programmable data processing apparatus to cause a series of operational steps to be performed on the computer or other programmable apparatus to produce a computer implemented process such that the instructions which execute on the computer or other programmable apparatus provide steps for implementing the functions/acts specified in the flowchart and/or block diagram block or blocks.

It is to be understood that the functions/acts noted in the blocks may occur out of the order noted in the operational illustrations. For example, two blocks shown in succession may in fact be executed substantially concurrently or the blocks may sometimes be executed in the reverse order, depending upon the functionality/acts involved. Although some of the diagrams include arrows on communication paths to show a primary direction of communication, it is to be understood that communication may occur in the opposite direction to the depicted arrows.

Computer program code for carrying out operations of the concepts described herein may be written in an object oriented programming language such as Java® or C++. However, the computer program code for carrying out operations of the disclosure may also be written in conventional procedural programming languages, such as the “C” programming language. The program code may execute entirely on the user's computer, partly on the user's computer, as a stand-alone software package, partly on the user's computer and partly on a remote computer or entirely on the remote computer. In the latter scenario, the remote computer may be connected to the user's computer through a local area network (LAN) or a wide area network (WAN), or the connection may be made to an external computer (for example, through the Internet using an Internet Service Provider).

Many different embodiments have been disclosed herein, in connection with the above description and the drawings. It will be understood that it would be unduly repetitious and obfuscating to literally describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

It will be appreciated by persons skilled in the art that the embodiments described herein are not limited to what has been particularly shown and described herein above. In addition, unless mention was made above to the contrary, it should be noted that all of the accompanying drawings are not to scale. A variety of modifications and variations are possible in light of the above teachings.

What is claimed is:

1. A hygiene monitoring method comprising:
determining when a hand washing event is required;
defining a healthcare area, a hygiene zone and a patient zone in which the hand washing event may occur;
determining a location of a user within at least one of the healthcare area, the hygiene zone and the patient zone;
transmitting data regarding the location of the user to a computing device; and
determining if the hand washing event has occurred within the healthcare area.

2. The method of claim 1, further comprising generating an alert if the hand washing event has not occurred.

3. The method of claim 1, wherein determining if the hand washing event has occurred within the healthcare area comprises:
determining if a soap dispenser is used while the user is within the healthcare area.

4. The method of claim 1, wherein determining if the hand washing event has occurred within the healthcare area comprises:
detecting the presence of hands of the user within the hygiene zone.

5. The method of claim 1, wherein determining if the hand washing event has occurred within the healthcare area comprises:
detecting the presence of an identification tag of the user for a predetermined amount of time.

6. A hygiene monitoring system comprising:
a set of customizable, pre-defined rules that determine when a hand washing event is required;
a geo-fence configured to determine perimeters of a healthcare area, a hygiene zone and a patient zone;
a location-generating mechanism configured to determine the location of the user within at least one of the healthcare area, hygiene zone and patient zone;
means for transmitting data regarding the location of the user to a computing device; and
means for determining if the soap dispenser was used by the user while within proximity of the soap dispenser.
